(19) United States
(12) Patent Application Publication
(10) Pub. No.: US 2025/0077848 A1
(43) Pub. Date: Mar. 6, 2025

(54) DEMONSTRATION UNCERTAINTY-BASED ARTIFICIAL INTELLIGENCE MODEL FOR OPEN INFORMATION EXTRACTION

(71) Applicant: NEC Laboratories America, Inc., Princeton, NJ (US)

(72) Inventors: Xujiang Zhao, Hillsborough, NJ (US); Haoyu Wang, Plainsboro, NJ (US); Zhengzhang Chen, Princeton Junction, NJ (US); Wei Cheng, Princeton Junction, NJ (US); Haifeng Chen, West Windsor, NJ (US); Yanchi Liu, Monmouth Junction, NJ (US); Chen Ling, Lawrenceville, NJ (US)

(21) Appl. No.: 18/817,793

(22) Filed: Aug. 28, 2024

Related U.S. Application Data
(60) Provisional application No. 63/536,532, filed on Sep. 5, 2023.

(51) Int. Cl. G06N 3/0475; G16H 80/00

(52) U.S. Cl. CPC G06N 3/0475; G16H 80/00

(57) ABSTRACT

Systems and methods for a demonstration uncertainty-based artificial intelligence model for open information extraction. A large language model (LLM) can generate initial structured sentences using an initial prompt for a domain-specific instruction extracted from an unstructured text input. Structural similarities between the initial structured sentences and sentences from a training dataset can be determined to obtain structurally similar sentences. The LLM can identify relational triplets from combinations of tokens from generated sentences using and the structurally similar sentences. The relational triplets can be filtered based on a calculated demonstration uncertainty to obtain a filtered triplet list. A domain-specific task can be performed using the filtered triplet list to assist the decision-making process of a decision-making entity.

### RELATED APPLICATION INFORMATION

This application claims priority to U.S. Provisional Application with number 63/536,532 filed on Sep. 5, 2023, incorporated herein by reference in its entirety.

### BACKGROUND

### Technical Field

The present invention relates to natural language processing with artificial intelligence models, and more particularly to a demonstration uncertainty-based artificial intelligence model for open information extraction.

### Description of the Related Art

Large language models (LLMs) have become ubiquitous due to the popularity of Chat-GPT™ in generating text as a trained LLM using large and diverse datasets. LLMs can perform general language tasks such as text generation, translation, etc., but can struggle to perform specific language tasks such as interpreting healthcare data.

### SUMMARY

According to an aspect of the present invention, a computer-implemented method for a demonstration uncertainty-based artificial intelligence (AI) model for open information extraction is provided, including, generating initial structured sentences with a large language model (LLM) using an initial prompt for a domain-specific instruction extracted from an unstructured text input, determining structural similarities between the initial structured sentences and sentences from a training dataset to obtain structurally similar sentences, identifying relational triplets from combinations of tokens from generated sentences using the LLM and the structurally similar sentences, filtering the relational triplets based on a calculated demonstration uncertainty to obtain a filtered triplet list, and performing a domain-specific task using the filtered triplet list to assist the decision-making process of a decision-making entity.

According to another aspect of the present invention, a system is provided, including, a memory device, one or more processor devices operatively coupled with the memory device to generate initial structured sentences from a large language model (LLM) using an initial prompt for a domain-specific instruction extracted from an unstructured text input, determine structural similarities between the initial structured sentences and sentences from a training dataset to obtain structurally similar sentences, identify relational triplets from combinations of tokens from generated sentences using the LLM and the structurally similar sentences, filter the relational triplets based on a calculated demonstration uncertainty to obtain a filtered triplet list, and perform a domain-specific task using the filtered triplet list to assist the decision-making process of a decision-making entity.

According to yet another aspect of the present invention, a non-transitory computer program product including a computer-readable storage medium that includes program code for a demonstration uncertainty-based artificial intelligence (AI) model for open information extraction is provided, wherein the program code when executed on a computer causes the computer to generate initial structured sentences from a large language model (LLM) using an initial prompt for a domain-specific instruction extracted from an unstructured text input, determine structural similarities between the initial structured sentences and sentences from a training dataset to obtain structurally similar sentences, identify relational triplets from combinations of tokens from generated sentences using the LLM and the structurally similar sentences, filter the relational triplets based on a calculated demonstration uncertainty to obtain a filtered triplet list, and perform a domain-specific task using the filtered triplet list to assist the decision-making process of a decision-making entity.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a high-level overview of a method for a demonstration uncertainty-based AI model for open information extraction is illustrated, in accordance with one embodiment of the present invention;

FIG. 2 is a block diagram showing a computing system implementing a demonstration uncertainty-based artificial intelligence model for open information extraction, in accordance with an embodiment of the present invention;

FIG. 3 is a block diagram illustrating a software implementation of a demonstration uncertainty-based artificial intelligence model for open information extraction, in accordance with an embodiment of the present invention;

FIG. 4 is a block diagram illustrating a practical application of a demonstration uncertainty-based artificial intelligence model for open information extraction, in accordance with an embodiment of the present invention; and

FIG. 5 is a block diagram illustrating deep learning neural networks for a demonstration uncertainty-based artificial intelligence model for open information extraction, in accordance with an embodiment of the present invention.

### DETAILED DESCRIPTION OF PREFERRED EMBODIMENTS

In accordance with embodiments of the present invention, systems and methods are provided for a demonstration uncertainty-based artificial intelligence model for open information extraction.

In an embodiment, demonstration uncertainty-based fine-tuning can improve the accuracy of artificial intelligence models for open information extraction. To perform demonstration uncertainty-based fine-tuning, a large language model (LLM) can generate initial structured sentences using an initial prompt for a domain-specific instruction extracted from an unstructured text input. Structural similarities between the initial structured sentences and sentences from a training dataset can be determined to obtain structurally similar sentences. The LLM can identify relational triplets from combinations of tokens from generated sentences and the structurally similar. A calculated demonstration uncertainty can filter relational triplets to obtain a filtered triplet list.

Performing domain-specific tasks with the filtered triplet list can assist the decision-making process of a decision-making entity. The domain-specific task can include sending a simplified text form of a medical diagnosis spoken by a health practitioner during a telehealth visit using the filtered triplet list that includes a patient's sickness, a medicine to treat the sickness, and the side effects of the medicine. In another embodiment, the domain-specific task can include controlling a vehicle based on a filtered triplet list that includes the starting point, vehicle instruction, and ending point.

The present embodiments can improve existing LLMs by enhancing the accuracy of the generated sentences of the LLM in relation to a domain-specific task. Because of this, the efficiency and accuracy of the applications using the LLMs, such as healthcare data summarization, vehicle control, and system control, are subsequently improved.

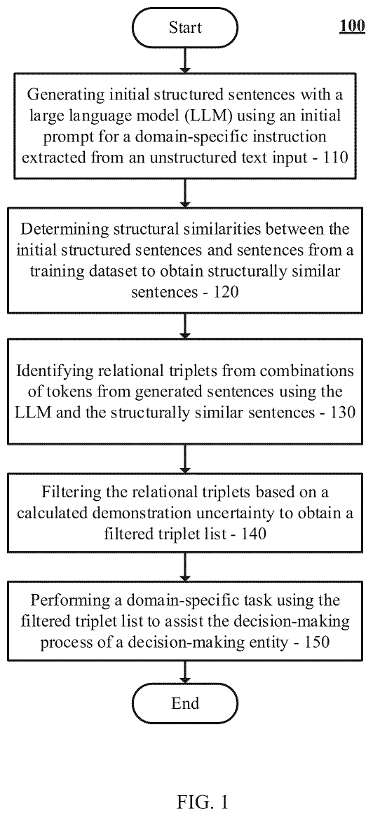

Referring now in detail to the figures in which like numerals represent the same or similar elements and initially to FIG. 1, a high-level overview of a method for a demonstration uncertainty-based AI model for open information extraction is illustrated, in accordance with one embodiment of the present invention.

In an embodiment, a demonstration-uncertainty based AI model for open information extraction can perform domain-specific tasks to assist the decision-making process of a decision-making entity. To obtain the demonstration-uncertainty based AI model for open information extraction, unstructured text data such as an input sentence with a subject, an action and an object can be collected. An initial prompt that guides the LLM step-by-step can generate initial structured sentences using a LLM with the collected text data. Structural similarities between the initial structured sentences and sentences from a training dataset can be determined and obtain structurally similar sentences. The LLM can identify relational triplets from a combination of structurally similar sentences and tokens from generated sentences relational triplets. A calculated demonstration uncertainty can filter relational triplets to obtain a filtered triplet list. Performing a domain-specific task using the filtered triplet list can assist the decision-making process of a decision-making entity.

In block 110, a large language model (LLM) can generate initial structured sentences using an initial prompt for a domain-specific instruction extracted from an unstructured text input.

In an embodiment, domain-specific instructions can guide the model to navigate the complexity and ambiguity of unstructured text input and output structured responses for the task. The domain-specific instruction can include a chain of instructions that give step-by-step instructions to finish a task. Initial structured sentences can include a series of text generated by the LLM in response to the initial prompt.

The unstructured text input can include a sequence of tokens/words \tilde{s}=<w₁, w₂, . . . , wₙ> that is written in prose. In another embodiment, the input can include an audio or video recording of an unstructured text input. The LLM can be trained to convert speech into text. The LLM can include autoregressive generation models, such as GPT™, Codex™, or Text-To-Text Transfer Transformer (T5).

The initial prompt can include the domain-specific instruction. The domain-specific instruction can include action items that an LLM can generate. The domain-specific task can include the target output so that the LLM has an action goal. A prompt generator can generate the initial prompts. The prompt generator can pull prompt templates from a database which varies depending on the domain. The prompt generator can use an LLM to generate the prompt templates based on a learned context of the domain-specific instruction.

For example, the domain can relate to healthcare and the initial prompt can include “Identify all combinations of subjective, action, objective, and possible adverbials for any given sentences, and present them in the form of triplets: (subjective, action, objective).” A healthcare dataset can provide the input sentences that can include medical diagnosis information: sickness, medicine, effect of medicine, instructions for taking the medicine, etc. The input sentence can include “You have cough, so you can take guaifenesin every six hours for five days.” The initial structured sentences from the input sentence can include “<cough, guaifenesin, take every six hours for five days>.”

In block 120, structural similarities between the initial structured sentences and sentences from a training dataset can be determined to obtain structurally similar sentences.

In an embodiment, to ensure that the LLM generates responses consistent with the domain-specific instruction, the present embodiments can incorporate few-shot examples sampled from the training set that are similar to the target sentence \tilde{s}. Specifically, given a training set S consisting of annotated sentences, the present embodiments can retrieve a small subset Ŝ⊆S of structurally similar sentences with the target sentence \tilde{s}.

A database linked to the prompt templates from a domain dataset can provide the few-shot examples based on the learned context of the LLM. Structural similarity can be computed using cosine similarity between sentence latent embeddings of the initial sentences and the few-shot examples. In another embodiment, the LLM can learn the structural similarity.

For example, if the target sentence \tilde{s} is a question with an attributive clause: “Who is the author who wrote the book that won the Pulitzer Prize last year?,” then the structurally-similar sentences from the training set can include: “Where is the restaurant that serves the best sushi in town?.”

In block 130, the LLM can identify relational triplets from combinations of tokens from generated sentences and the structurally similar sentences.

In an embodiment, the LLM can identify relational triplets from combinations of tokens from generated sentences and the structurally similar sentences. Relational triplets can include a group of words that can have a predicate, a subjective entity and an objective entity. The predicate can include a relationship between the subjective entity and the objective entity (e.g., action). The subjective entity can include the phrase that represents the entity to be acted upon (e.g., subject). The objective entity can include the phrase that will be affected by the predicate with the subjective entity (e.g., object). In another embodiment, the relational triplets can include a different combination of entities based on a learned context of the domain. For example, the relational triplets for time-series data can include a starting point, ending point, and relevance.

T=[T₁, T₂, . . . ] with the i-th triplet Tᵢ=<wₛ, pᵢ, wₒ> representing a fact in the source sequence, where p denotes the predicate in Tᵢ, wₛ and wₒ are the subjective and objective entities of Tᵢ respectively.

Tokens are words or phrases that are taken from an input sentence. The LLM can extract tokens from the input sentences. The tokens are combined with each other based on the learned context of the LLM depending on the domain-specific task.

In block 140, a calculated demonstration uncertainty can filter the relational triplets to obtain a filtered triplet list.

In an embodiment, the present embodiments can iteratively allow LLM to generate answers of the target sentence \tilde{s} by employing [symbol] as an in-context learning example and fine tune the LLM to adapt to the domain it was trained for. The present embodiments can use the prompt to maximize the generated combinations of tokens from an input sentence. For example, the prompt can include “Identify as many combinations as possible in the following sentence: \tilde{s}.”

The present embodiments can collect all the generated relational triplets from each sampled [symbol] into a list T_{\tilde{s}} and calculate the Demonstration Uncertainty U_{\tilde{S}} of each Tᵢ∈T_{\tilde{S}}

$${U}_{\hat{s}} = \{ {u}_{i} \vert 1 - \frac{1}{N} {\sum }_{j = 1}^{N} {{T}_{j} = {T}_{i}}_{} \} ,$$

where N denotes the total number of elements in T_{\tilde{S}} and [symbol] is an indicator function counting each element's occurrence. Intuitively, if the uncertainty score uᵢ is high, then it denotes the corresponding Tᵢ appears less frequent in T_{\tilde{S}}, and vice versa.

The present embodiments can adopt a threshold k to filter high uncertain Tᵢ, i.e. T_{\tilde{S}}={Tᵢ|uᵢ≥k} by eliminating relational triplets below the threshold. Finally, based on the feedback from LLM, the present embodiments can form the list of triplets Tᵢ as the output. The threshold can range from zero to one. For example, the threshold can include 0.5.

In block 150, performing a domain-specific task using the filtered triplet list can assist the decision-making process of a decision-making entity.

In an embodiment, the present embodiments can perform a domain-specific task using the filtered triplet list which can assist the decision-making process of a decision-making entity. In an embodiment, the domain-specific task can include sending a simplified text form of a medical diagnosis spoken by a health practitioner through a network during a telehealth visit using the filtered triplet list that includes a patient's sickness, a medicine to treat the sickness, and the side effects of the medicine. The simplified text form can include audio converted from text.

In another embodiment, the domain-specific task can include constructing a medical knowledge base using the filtered triplet list that is based on medical knowledge newly discovered, obtained, or updated by a healthcare entity (e.g., health organization, hospital, etc.). The healthcare knowledge base can include global knowledge such as global epidemic data. The healthcare knowledge base can include localized knowledge such as hospitalization rates within a community. The healthcare knowledge base can be continuously learned and constructed by using newly acquired data and feedback obtained from domain experts (e.g., trained LLM for the domain, etc.). The filtered triplet list can ensure and improve the accuracy of the medical knowledge base constructed.

In another embodiment, the domain-specific task can include controlling a vehicle based on a filtered triplet list that includes the starting point, vehicle instruction, and ending point. In another embodiment, the domain-specific task can include controlling a system, such as a cloud system, based on a filtered triplet list obtained from system logs or system instructions that includes a system entity (e.g., node, container, task, workload, etc.), system action (e.g., increase, decrease, block, etc.), and system metric (e.g., bandwidth, processor, etc.)

The present embodiments can improve existing LLMs by enhancing the accuracy of the generated sentences of the LLM in relation to a domain-specific task. Because of this, the efficiency and accuracy of the applications using the LLMs, such as healthcare data summarization, vehicle control, and system control, are subsequently improved.

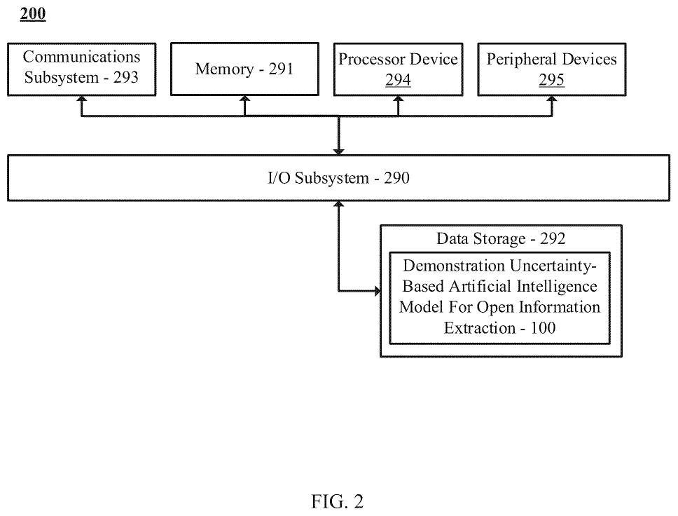

Referring now to FIG. 2, a block diagram showing a computing system demonstration uncertainty-based artificial intelligence model for open information extraction, in accordance with an embodiment of the present invention.

The computing device 200 illustratively includes the processor device 294, an input/output (I/O) subsystem 290, a memory 291, a data storage device 292, and a communication subsystem 293, and/or other components and devices commonly found in a server or similar computing device. The computing device 200 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 291, or portions thereof, may be incorporated in the processor device 294 in some embodiments.

The processor device 294 may be embodied as any type of processor capable of performing the functions described herein. The processor device 294 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 291 may be embodied as any type of volatile or non-volatile memory or data storage capable of performing the functions described herein. In operation, the memory 291 may store various data and software employed during operation of the computing device 200, such as operating systems, applications, programs, libraries, and drivers. The memory 291 is communicatively coupled to the processor device 294 via the I/O subsystem 290, which may be embodied as circuitry and/or components to facilitate input/output operations with the processor device 294, the memory 291, and other components of the computing device 200. For example, the I/O subsystem 290 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 290 may form a portion of a system-on-a-chip (SOC) and be incorporated, along with the processor device 294, the memory 291, and other components of the computing device 200, on a single integrated circuit chip.

The data storage device 292 may be embodied as any type of device or devices configured for short-term or long-term storage of data such as, for example, memory devices and circuits, memory cards, hard disk drives, solid state drives, or other data storage devices. The data storage device 292 can store program code for a demonstration uncertainty-based artificial intelligence model for open information extraction 100. Any or all of these program code blocks may be included in a given computing system.

The communication subsystem 293 of the computing device 200 may be embodied as any network interface controller or other communication circuit, device, or collection thereof, capable of enabling communications between the computing device 200 and other remote devices over a network. The communication subsystem 293 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 200 may also include one or more peripheral devices 295. The peripheral devices 295 may include any number of additional input/output devices, interface devices, and/or other peripheral devices. For example, in some embodiments, the peripheral devices 295 may include a display, touch screen, graphics circuitry, keyboard, mouse, speaker system, microphone, network interface, and/or other input/output devices, interface devices, GPS, camera, and/or other peripheral devices.

Of course, the computing device 200 may also include other elements (not shown), as readily contemplated by one of skill in the art, as well as omit certain elements. For example, various other sensors, input devices, and/or output devices can be included in computing device 200, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing system 200 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid-state memory, magnetic tape, a removable computer diskette, a random-access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

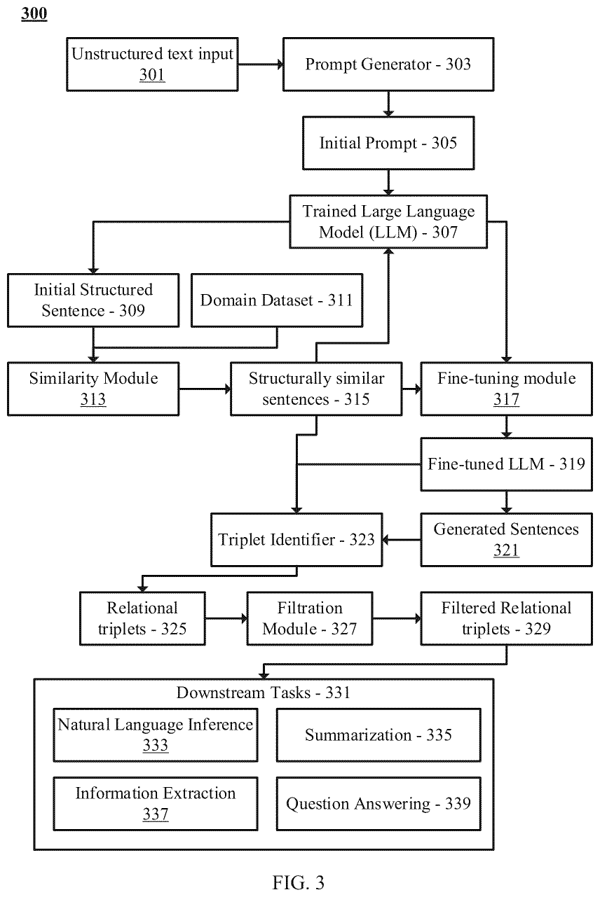

Referring now to FIG. 3, a block diagram illustrating a software implementation of demonstration uncertainty-based artificial intelligence model for open information extraction, in accordance with an embodiment of the present invention.

In an embodiment, a system including the software implementation of the demonstration uncertainty-based LLM for open information extraction 300 is described. A prompt generator 303 can process unstructured text input 301 to obtain an initial prompt 305. A trained LLM 307 can generate initial structured sentences 309 based on the initial prompt 305. A similarity module 313 can process few shot examples from a domain dataset 311 and the initial structured sentences 309 to generate structurally similar sentences 315. A fine-tuning module 317 can fine tune the trained LLM 307 using the structurally similar sentences to obtain the fine-tuned LLM 319. The fine-tuned LLM 319 can obtain generated sentences 321 and can process the generated sentences 321 with the structurally similar sentences 315 by using a triplet identifier 323. The triplet identifier 323 can use the fine-tuned LLM 319 to obtain relational triplets 325. A filtration module 327 can filter the relational triplets 325 based on a calculated demonstration uncertainty to obtain filtered relational triplets 329. The filtered relational triplets 329 can be used to perform downstream tasks 331 that can include natural language inference 333, summarization 335, information extraction 337, question answer 339, etc.

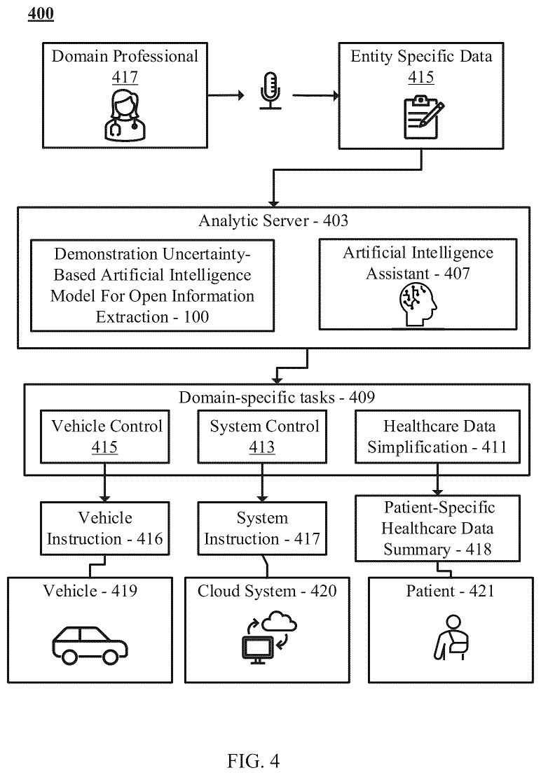

Referring now to FIG. 4, a block diagram illustrating a practical application of demonstration uncertainty-based artificial intelligence model for open information extraction, in accordance with an embodiment of the present invention.

In an embodiment, a system 400 performing domain-specific tasks with the filtered triplet list to assist the decision-making process of a decision-making entity is shown.

A domain professional 417 can provide domain instructions which can include an audio, video, or text. The domain instruction can include entity specific data 415. The entity specific data 415 can include text that is relevant to the domain task at hand and is specific to an entity. The entity can include a person, organization, etc., that the domain professional 417 is trying to help. The analytic server 403 can receive entity specific data 415 through a network. The analytic server 403 can include the demonstration uncertainty-based artificial intelligence model for open information extraction 100 and an artificial intelligence assistant 407 that uses the demonstration uncertainty-based artificial intelligence model for open information extraction 100. The analytic server can send performed domain-specific tasks 409 to the entity. The domain specific tasks can include healthcare data simplification 411, system control 413 and vehicle control 415. Other domain specific tasks are contemplated.

In an embodiment, healthcare data simplification 411 can include sending a simplified text form of a medical diagnosis (e.g., entity specific data 415) spoken by a domain professional 417 (e.g., health practitioner) during a telehealth visit using the filtered triplet list that includes a patient's 421 sickness, a medicine to treat the sickness, and the side effects of the medicine. The simplified text form of the medical diagnosis can be generated in real time, sent through a network, and shown to the patient 421 as a generated caption while the health practitioner is discussing the medical diagnosis to the patient 421. In another embodiment, the simplified text form can include a table, graph, or chart. The simplified text form can include audio converted from text.

In another embodiment, the domain-specific task can include constructing a medical knowledge base using the filtered triplet list that is based on medical knowledge newly discovered, obtained, or updated by a healthcare entity (e.g., health organization, hospital, etc.). The healthcare knowledge base can include global knowledge such as global epidemic data. The healthcare knowledge base can include localized knowledge such as hospitalization rates within a community.

In another embodiment, vehicle control 415 can include controlling a vehicle 419 based on a filtered triplet list that includes the starting point, vehicle instruction, and ending point. The vehicle 419 can include a drone, car, truck, etc. The vehicle instruction can include stopping, moving, changing direction, etc. A vehicle control system within a vehicle that converts instructions to vehicle movement can implement the vehicle control 415.

In another embodiment, the system control 413 can include controlling a system, such as a cloud system 420, based on a filtered triplet list obtained from system logs or system instructions that includes a system entity (e.g., node, container, task, workload, etc.), system action (e.g., increase, decrease, block, etc.), and system metric (e.g., bandwidth, processor, etc.). A cloud system 420 that converts instructions to cloud system updates can implement the system control 413.

Other practical applications are contemplated.

The present embodiments use a trained LLM and fine-tuned LLM to generate a filtered triplet list which transforms unstructured text into structured text. The trained LLM and fine-tuned LLM can include deep learning neural networks.

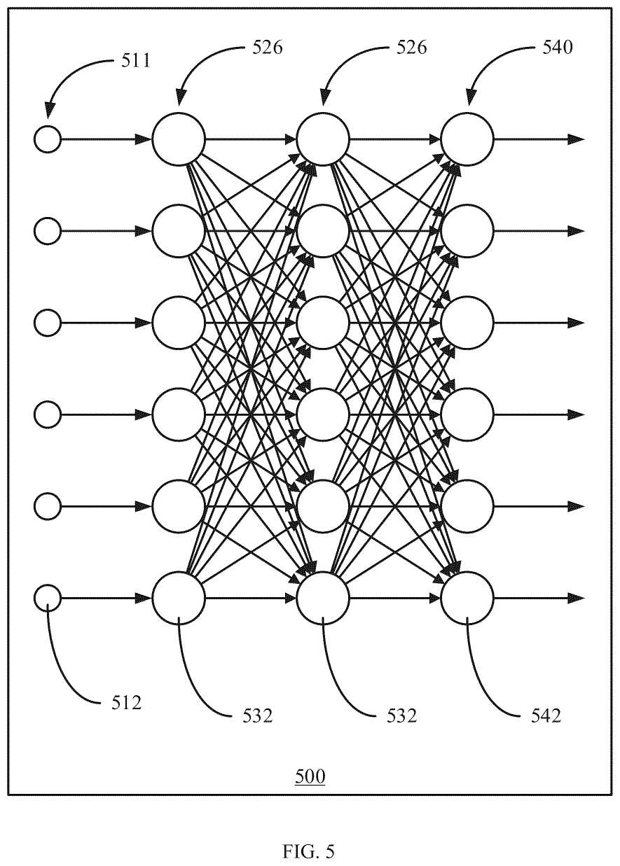

Referring now to FIG. 5, a block diagram illustrating deep learning neural networks for demonstration uncertainty-based artificial intelligence model for open information extraction, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 500, such as a multilayer perceptron, can have an input layer 511 of source neurons 512, one or more computation layer(s) 526 having one or more computation neurons 532, and an output layer 540, where there is a single output neuron 542 for each possible category into which the input example could be classified. An input layer 511 can have a number of source neurons 512 equal to the number of data values 512 in the input data 511. The computation neurons 532 in the computation layer(s) 526 can also be referred to as hidden layers, because they are between the source neurons 512 and output neuron(s) 542 and are not directly observed. Each neuron 532, 542 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, Wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

In an embodiment, the computation layers 526 of the trained LLM 307 can learn relationships and context between the initial generated sentences and the few shot examples from the domain dataset 311. The output layer 540 of the trained LLM 307 can then provide the overall response of the network as a likelihood score of a prediction of the relationship and context between the initial generated sentences and the few shot examples from the domain dataset 311. In another embodiment, the trained LLM 307 can output generated text based on the initial prompt 305 to generate initial structured sentences. In another embodiment, the trained LLM 307 can identify structural similarity between the initial generated sentences and the few shot examples from the domain dataset 311 based on the learned context.

Training a deep neural network can involve two phases, a forward phase where the weights of each neuron are fixed and the input propagates through the network, and a backwards phase where an error value is propagated backwards through the network and weight values are updated.

The computation neurons 532 in the one or more computation (hidden) layer(s) 526 perform a nonlinear transformation on the input data 512 that generates a feature space. The classes or categories may be more easily separated in the feature space than in the original data space.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for a demonstration uncertainty-based artificial intelligence model for open information extraction, comprising:
generating initial structured sentences with a large language model (LLM) using an initial prompt for a domain-specific instruction extracted from an unstructured text input;
determining structural similarities between the initial structured sentences and sentences from a training dataset to obtain structurally similar sentences;
identifying relational triplets from combinations of tokens from generated sentences using the LLM and the structurally similar sentences;
filtering the relational triplets based on a calculated demonstration uncertainty to obtain a filtered triplet list; and
performing a domain-specific task using the filtered triplet list to assist the decision-making process of a decision-making entity.

2. The computer-implemented method of claim 1, wherein performing a domain-specific task further comprises sending, through a network, a simplified form of a medical diagnosis spoken by a healthcare practitioner during a telehealth visit using the filtered triplet list that includes a patient's sickness, a medicine to treat the sickness, and the side effects of the medicine.

3. The computer-implemented method of claim 1, wherein performing a domain-specific task further comprises controlling a vehicle based on a filtered triplet list that includes a starting point, a vehicle instruction, and an ending point.

4. The computer-implemented method of claim 1, wherein generating initial structured sentences further comprises generating the initial prompt as a chain of instructions to guide the LLM step-by-step generated using a prompt template.

5. The computer-implemented method of claim 1, wherein determining structural similarities further comprises computing cosine similarity between sentence latent embeddings of the initial structured sentences and sentences from a training dataset.

6. The computer-implemented method of claim 1, wherein identifying relational triplets further comprises iteratively generating sentences that answers a target sentence by using the sampled structurally similar sentences as an in-context learning example.

7. The computer-implemented method of claim 1, wherein filtering the relational triplets further comprises eliminating generated relational triplets having a demonstration-uncertainty above a threshold.

8. A system, comprising:
a memory device;
one or more processor devices operatively coupled with the memory device to:
generate initial structured sentences from a large language model (LLM) using an initial prompt for a domain-specific instruction extracted from an unstructured text input;
determine structural similarities between the initial structured sentences and sentences from a training dataset to obtain structurally similar sentences;
identify relational triplets from combinations of tokens from generated sentences using the LLM and the structurally similar sentences;
filter the relational triplets based on a calculated demonstration uncertainty to obtain a filtered triplet list; and
perform a domain-specific task using the filtered triplet list to assist the decision-making process of a decision-making entity.

9. The system of claim 8, wherein one or more processor devices operatively coupled with the memory device to perform a domain-specific task further comprises to send, through a network, a simplified form of a medical diagnosis spoken by a health practitioner during a telehealth visit using the filtered triplet list that includes a patient's sickness, a medicine to treat the sickness, and the side effects of the medicine.

10. The system of claim 8, wherein one or more processor devices operatively coupled with the memory device to perform a domain-specific task further comprises to control a vehicle based on a filtered triplet list that includes the starting point, vehicle instruction, and ending point.

11. The system of claim 8, wherein one or more processor devices operatively coupled with the memory device to generate initial structured sentences further comprises to generate the initial prompt as a chain of instruction to guide the LLM step-by-step generated using a prompt template.

12. The system of claim 8, wherein one or more processor devices operatively coupled with the memory device to determine structural similarities further comprises computing cosine similarity between sentence latent embeddings of the initial structured sentences and sentences from a training dataset.

13. The system of claim 8, wherein one or more processor devices operatively coupled with the memory device to identify relational triplets further comprises to iteratively generate sentences that answers a target sentence by using the sampled structurally similar sentences as an in-context learning example.

14. The system of claim 8, wherein one or more processor devices operatively coupled with the memory device to filter the relational triplets further comprises to eliminate generated relational triplets having a demonstration-uncertainty above a threshold.

15. A non-transitory computer program product comprising a computer-readable storage medium including program code for a demonstration uncertainty-based artificial intelligence model for open information extraction, wherein the program code when executed on a computer causes the computer to:
generate initial structured sentences from a large language model (LLM) using an initial prompt for a domain-specific instruction extracted from an unstructured text input;
determine structural similarities between the initial structured sentences and sentences from a training dataset to obtain structurally similar sentences;
identify relational triplets from combinations of tokens from generated sentences using the LLM and the structurally similar sentences;
filter the relational triplets based on a calculated demonstration uncertainty to obtain a filtered triplet list; and
perform a domain-specific task using the filtered triplet list to assist the decision-making process of a decision-making entity.

16. The non-transitory computer program product of claim 15, wherein to perform a domain-specific task further comprises to control a vehicle based on a filtered triplet list that includes the starting point, vehicle instruction, and ending point.

17. The non-transitory computer program product of claim 15, wherein to generate initial structured sentences further comprises to generate the initial prompt as a chain of instruction to guide the LLM step-by-step generated using a prompt template.

18. The non-transitory computer program product of claim 15, wherein to determine structural similarities further comprises computing cosine similarity between sentence latent embeddings of the initial structured sentences and sentences from a training dataset.

19. The non-transitory computer program product of claim 15, wherein to identify relational triplets further comprises iteratively generating sentences that answers a target sentence by using the sampled structurally similar sentences as an in-context learning example.

20. The non-transitory computer program product of claim 15, wherein to filter the relational triplets further comprises eliminating generated relational triplets having a demonstration-uncertainty above a threshold.
